(19) United States
(12) Patent Application Publication
(10) Pub. No.: US 2025/0055221 A1
(43) Pub. Date: Feb. 13, 2025

(54) IMPLANTABLE ELECTRICAL CONNECTOR

(71) Applicant: Berlin Heart GmbH, Berlin (DE)

(72) Inventors: Michael Wange, Berlin (DE); Konstantin Austilat, Berlin (DE)

(73) Assignee: Berlin Heart GmbH, Berlin (DE)

(21) Appl. No.: 18/797,881

(22) Filed: Aug. 8, 2024

(30) Foreign Application Priority Data
Aug. 9, 2023 (EP) 23 190 667.8

(51) Int. Cl. H01R 13/52; H01R 13/24; H01R 13/627; H01R 24/20; H01R 24/28; H01R 24/58; H01R 107/00

(52) U.S. Cl. CPC H01R 13/5224; H01R 13/2407; H01R 13/2471; H01R 13/521; H01R 13/5216; H01R 13/5219; H01R 13/6273; H01R 24/20; H01R 24/28; H01R 24/58; H01R 2107/00; H01R 2201/12

(57) ABSTRACT

The invention relates to an implantable electrical plug connector with two plug connector parts which can be joined together to make electrical contact, one of which is connected to a first line and the other of which is connected to a second line, wherein each plug connector part has at least one or at least two contact pole parts respectively in the form of a contact pin or a contact socket, which can each be plugged together with the contact pole parts of the other plug connector part to make contact, wherein the longitudinal axes of the contact pole parts (plugging direction of the contact pole parts in which they are moved to establish the plug-in connection) extend at an angle of at least 30 degrees, in particular at an angle of 90 degrees, to a longitudinal axis of the first and/or second plug connector part.

### PRIORITY

This application claims priority to EP Application No. 23 190 667.8, filed on Aug. 9, 2023, the entirety of which is fully incorporated by reference herein.

### TECHNICAL FIELD

The invention relates to the field of medical technology, mechanical engineering and electrical engineering and can be used to particular advantage in devices and appliances which can be implanted into the body of a patient.

### BACKGROUND

Various devices are known in the field of medical technology which can be implanted fully or partially into the body of a patient, such as for example heart pumps, pacemakers, defibrillators and similar devices. Many such devices require an electrical power supply, are controlled electrically or provide data which is transmitted electrically. Therefore, in many cases the devices have to be connected via electrical lines or connected to other units. As a relatively large number of insulated individual conductors are required for many such connections, relatively complex connectors are used to create detachable connections. Multi-pole connectors with parallel connector poles, such as those described in US patent U.S. Pat. No. 10,265,450B2, or so-called multi-channel in-line connectors, which essentially have several coaxial contacts arranged one behind the other along a plug pin or socket, are known for this purpose. Such connectors are common in medical technology and are also standardized in some cases.

If the number of individual contacts required exceeds certain limits, the plug pins and sockets become longer and longer, so that the connectors constructed in this way become unusable during implantation due to their space requirements.

In view of the prior art, the present invention is based on the task of creating a plug connector which also offers a simple and secure plug connection for a plurality of conductors which takes up as little space as possible.

The problem is solved by the features of the invention according to any one of claims 1 and 2.

### SUMMARY

Accordingly, the invention relates to an implantable electrical plug connector with two plug connector parts that can be joined together to make electrical contact, one of which is connected to a first line and the other of which is connected to a second line, wherein each plug connector part comprises at least one or at least two contact pole parts in the form of a contact pin or a contact socket respectively, which can each be plugged together with contact pole parts of the other plug connector part to make contact,

wherein the longitudinal axes of the contact pole parts extend at an angle of at least 30 degrees, in particular at an angle of 90 degrees, to a longitudinal axis of the first and/or second plug connector part. The longitudinal axes of the contact pole parts define the plug-in direction of the contact pole parts in which they are joined together to create the plug connection. The longitudinal axis of a plug connector part can be defined by a direction of the largest longitudinal extension of a plug connector part or, for example if a plug connector part has a cylindrical housing, by the cylinder axis of the housing. The angle formed by the longitudinal axes of the contact pole parts and at least one of the longitudinal axes of a plug connector part, which is at least 30 degrees, is the smaller of the two angles formed by the said axes, so that when the plug connector parts are moved when being plugged in plug-in direction along a longitudinal axis of the contact pole parts the two plug connector parts also move nearer to one another in axial direction if the angle is less than 90 degrees.

The fact that the longitudinal axes of the contact pole parts form an angle of at least 30 degrees, in particular 90 degrees, with a longitudinal axis of the first and/or the second plug connector part, means that significantly less space is required when plugging and unplugging the plug connector than in the known arrangement in which the longitudinal axes of the connector poles extend parallel to the longitudinal axis of one or more plug connector parts. In the latter case, the lengths of the contact pole parts and parts of the plug connector housing as well as the line connections add up in the longitudinal direction in the position before the plug connector parts are plugged together and after they are unplugged so that the space required significantly exceeds the available space in a patient's body. This applies in particular to the use of such plug connectors in children.

The invention further relates to an implantable electrical plug connector with two plug connector parts which can be joined together to make electrical contact, one of which is connected to a first line and the other of which is connected to a second line, wherein each plug connector part has at least one or at least two contact pole parts each in the form of a contact pin or a contact socket, which can each be plugged together with contact pole parts of the other plug connector part to make contact, wherein the longitudinal axes of the contact pole parts extend at an angle of at least 30 degrees, in particular at an angle of 90 degrees, to a longitudinal extension direction of the first and/or the second line.

In this context, the longitudinal extension direction of the first and second line is defined as the longitudinal extension direction of the respective line directly in the connection region to the respective plug connector part, or also the outlet direction of the respective line from a plug connector part. Also in this case, the longitudinal axes of the contact pole parts are parallel to the plug-in direction of the contact pole parts in which they are joined together to form the plug connection.

The angle formed by the longitudinal axes of the contact pole parts and at least one of the longitudinal extension directions of the first and/or second line and which is at least 30 degrees, is the smaller of the two angles formed by the said axes/directions, so that when the plug connector parts are moved in plug-in direction along a longitudinal axis of the contact pole parts the two plug connector parts also move closer to one another in axial direction if the angle is less than 90 degrees.

In many cases, the space required for a plug connector of this type can also be at least partly determined by the line connections and the connected lines.

In this case, in particular if at least one of the lines is not connected parallel to a longitudinal axis of the respective plug connector, i.e. in the extension of this longitudinal axis, it can be advantageous if the plug-in direction of the plug connector and the direction of the longitudinal axes of the contact pole parts are not parallel to the longitudinal direction defined by the line connections, i.e. in particular to a longitudinal extension direction of the first and/or second line, but at an angle of at least 30 degrees to this direction.

In one particular embodiment of the invention it can be provided that at least two, in particular at least three, further particularly at least four contact pole parts are provided next to each other on each of the plug connector parts, which have longitudinal axes parallel to one another.

By distributing the various contact connections of separate, electrically insulated lines to different contact poles, each of the contact poles has a smaller number of contacts, so that the contact poles and thus the contact pole parts, i.e. the contact pins and the sockets, can have a shorter overall length in plug-in direction, so that the plug connector takes up less space when the contacts are made and disconnected.

In a further embodiment of the invention it can be provided that at least one of the contact pole parts has a plurality of concentric contacts that are electrically insulated from one another.

Such contact poles with concentric contacts can be constructed in a particularly space-saving manner.

Such contact poles can be configured to be particularly space-saving in that at least two of the concentric contacts are offset from each other in the longitudinal direction of the respective contact pole part. In this sense, more than two, in particular more than three or more than four contacts can also be offset from one another in the longitudinal direction on one or more of the contact poles. This can also be case for example for all contact poles of a plug connector.

It is a particular advantage that the contact pins or the contact sockets each have one or more annular spring contacts.

Such annular spring contacts each have the form of a torus which is formed by a coil spring. In the plugged-in state of the plug connection, the annular springs are elastically compressed in radial direction between a contact pin and a socket and in this way establish the electrically conductive contact between them.

Instead of annular spring contacts, also other, preferably resilient, contact elements can also be provided between a contact pin and a contact socket and distributed in their longitudinal direction.

In a plug connector according to the invention, it can also be provided advantageously that a sealing ring is arranged between two contacts in the direction of the longitudinal axes of the contact pole parts.

These sealing rings can be configured as elastomer rings and can for example be arranged between two annular spring contacts. The sealing rings can thereby be positioned along a contact pole so that they lie in grooves of a contact pin or a contact socket. The sealing rings prevent body fluids, which enter the interior of the plug connector in the implanted state despite other sealing measures, from bridging the gap between two contacts at a contact pole and thereby creating a short circuit or making an undesirable contact.

In the case of an electrical plug connector with concentric contacts, it can also be provided that contact pole parts that engage with each other when plugged together are configured in such a way that one or more sealing rings, which are assigned to one of the contact pole parts, are in sealing contact with sealing surfaces of the other respective contact pole part, whereby the sealing surfaces are formed in particular by cylindrical surfaces of electrically insulating sleeves.

This means that sealing rings can be arranged on one of the contact pole parts at specific, defined distances from one another, while on the other, complementary contact pole part there are sealing surfaces at corresponding distances between contacts, against which the sealing rings bear in a fluid-tight manner when the plug connection is plugged together.

The invention can be further characterized in that the two connector parts are held together by at least one elastic ring embracing both plug connector parts and/or a latching connection that engages in the direction of the longitudinal axes of the contact pole parts.

The connector parts can be held together in a space-saving manner by such retaining or fastening elements, whereby only minimal space is required for fastening and releasing the fastening elements.

A further embodiment of the invention can provide that each pair of contact pole parts, when plugged together, is surrounded by an elastic contact pole sealing ring which is arranged between the first plug connector part and the second plug connector part and bears in a sealing manner against each of the plug connector parts, wherein it is provided in particular that the contact pole sealing rings are each integrated into one of the plug connector parts.

Such a contact pole sealing ring bears fluid-tight against both plug connector parts and can surround one or more contact poles respectively. This prevents the ingress of fluids to the two contact pole parts when the plug connection is plugged together. The contact pole sealing ring thus represents a fluid barrier that is ahead of the sealing rings that are arranged between the contacts.

Furthermore, the invention can also relate to an implantable plug connector of the type described above, wherein the longitudinal axes of the contact pole parts extend at an angle of at least 30 degrees, in particular at an angle of 90 degrees, to a longitudinal axis of the first and/or the second plug connector part or also parallel to the longitudinal axis of the first and/or the second plug connector part, wherein one of the plug connector parts, which has at least one contact socket, comprises or bears on its side facing the other plug connector part when the plug connector is plugged together a sealing sleeve which is clastically deformable at least in certain regions, in particular with at least one opening for receiving a contact pin.

In addition to other sealing measures, such a sealing sleeve can protect the contact pole parts from the ingress of liquids that could cause faulty contacts or short circuits. Due to the at least partially elastic design, the sealing sleeve can effectively bear fluid-tight against other components, at least in certain areas. When the plug connector is plugged together, the sealing sleeve lies between the two plug connector parts in the axial direction and/or plug-in direction of the plug connector.

The plug connector can be advantageously configured in that the sealing sleeve is made of an elastomer, in particular a biocompatible elastomer.

In this case, the sealing sleeve can be manufactured homogeneously as a whole and form sealing surfaces on its entire surface, including for sealing on a contact pin inserted into an opening.

A further embodiment of the plug connector can provide that one or more openings of the sealing sleeve each open into a channel for receiving a contact pin, wherein the channels run respectively coaxially to contact sockets of the plug connector part which comprises the scaling sleeve and wherein at least one of the channels has a narrow point in the region of its opening.

In the plugged-in state of the plug connector, contact pins are inserted into the openings and through the channels in the sealing sleeve and the channels can be sealed at the narrow points, in that the diameter of the narrow points is smaller than the diameter of the contact pins, at least in a compressed state of the sealing sleeve in axial direction, and in that the material of the sealing sleeve is elastically expanded by the contact pins in the region of the narrow points and bears fluid-tight against the contact pins.

The connector can also be configured in such a way that the narrow points are formed by inwardly pointing sealing lips in the channels.

The plug connector can also be advantageously configured in such a way that the sealing sleeve in the plug connector can be compressed in axial direction or plug-in direction of the plug connector in such a way that it expands in the directions perpendicular to the axial direction or plug-in direction of the plug connector.

For example, in a manner known per se, the two connector parts can be pressed together in axial direction by a union nut or an elastic clamp, wherein the sealing sleeve is compressed in axial direction and expands in transverse direction. In this way for example the narrow points in the channels can narrow and tightly enclose the contact pins. As a result of the expansion the sealing sleeve can also touch in a fluid-tight manner against other parts of the plug connector on the outside of its circumference.

The invention is illustrated in the following by embodiments shown in Figures of a drawing and explained below.

In the Figures:

### BRIEF DESCRIPTION OF THE DRAWINGS

FIG. 1 shows a plug connector according to the prior art,

FIG. 2 shows a plug connector according to the invention in a disconnected state of the plug connector parts,

FIG. 3 shows the plug connector from FIG. 2 from a different perspective,

FIG. 4 shows a plug connector according to the invention in the connected state,

FIG. 5 shows two plug connector parts with an elastic ring and a latching connection before connecting,

FIG. 6 shows the elements of FIG. 5 after connecting,

FIG. 7 shows a longitudinal cross-section of two plug connector parts according to the invention,

FIG. 8 shows two plug connector parts, wherein the full length of the contact pins can be seen,

FIG. 9 shows the two plug connector parts of FIG. 8, wherein the contact pole sealing rings on the contact sockets are shown,

FIGS. 10, 11 show schematically the angles between the longitudinal axes of the contact pole parts and other axes of the plug connector, which are advantageous according to the invention,

FIG. 12 shows a plug connector with a sealing sleeve between the plug connector parts,

FIG. 13 shows an axial plug connector with a sealing sleeve and

FIG. 14 shows details of a sealing sleeve in a longitudinal cross-section and in front view.

### DETAILED DESCRIPTION

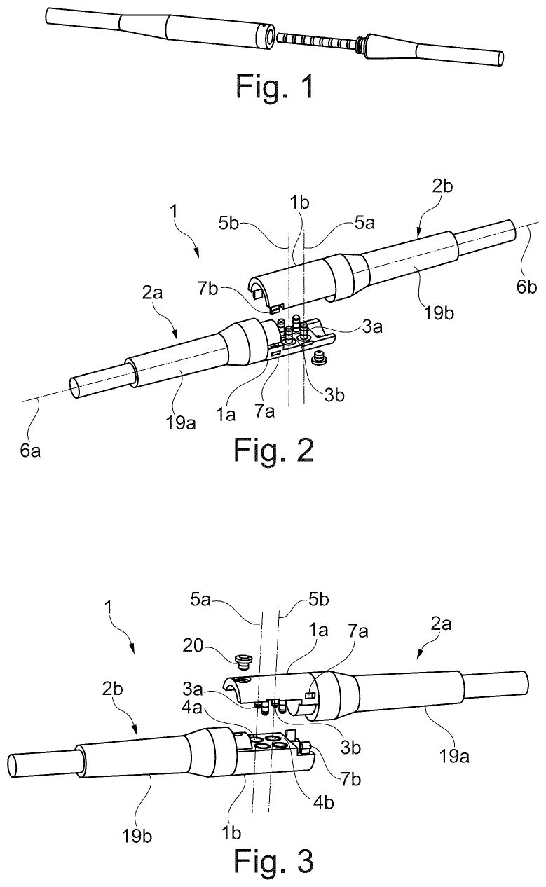

FIG. 1 shows a perspective view of a plug connector according to the prior art. A contact pin on the right-hand side of the drawing has several concentric electrical contacts offset from one another in the longitudinal direction of the contact pin, which interact with individual contacts inside the contact socket on the left-hand side of the drawing. A considerable amount of space is required in axial direction for disconnecting or plugging in the plug connection. If the number of contacts exceeds a certain limit, it can no longer be used as an implantable plug connector in a patient's body.

FIG. 2 shows a perspective view of two plug connector parts 1a, 1b in a disconnected state. The plug connector 1 is divided along a surface which extends parallel to the longitudinal axes 6a, 6b of the plug connector parts and the lines 2a, 2b. The plug connector part 1a shown in FIG. 2 at the bottom has contact pins 3a, 3b, the longitudinal axes 5a, 5b of which extend parallel to one another. The longitudinal axes 5a, 5b of the contact pins also extend perpendicular to the longitudinal axes 6a, 6b of the plug connector parts 1a, 1b. The two plug connector parts 1a, 1b are plugged together after an initial alignment in a direction parallel to the longitudinal axes 5a, 5b of the contact pins 3a, 3b and the contact sockets not shown in FIG. 2. A bend protection 19a, 19b attached to the respective plug connector part 1a, 1b is also assigned to each of the lines 2a, 2b as part of the lines.

A fastening screw 20 is also shown, as well as a latching connection, comprising two latching lugs 7b and associated latching openings 7a.

In principle, fastening the screw 20 perpendicular to the longitudinal axes 6a, 6b of the plug connector parts requires lateral access, while the latching connection snaps into place without further action when plugging the connector parts together. It may therefore be expedient to avoid or replace the fastening screw 20 in other embodiments.

FIG. 3 shows a perspective view of the plug connector parts 1a, 1b of FIG. 2 from a different direction. FIG. 3 shows the contact sockets 4a, 4b, into which the contact pins 3a, 3b are inserted along the longitudinal axes 5a, 5b. FIG. 3 also shows the latching connection 7a, 7b and the fastening screw 20.

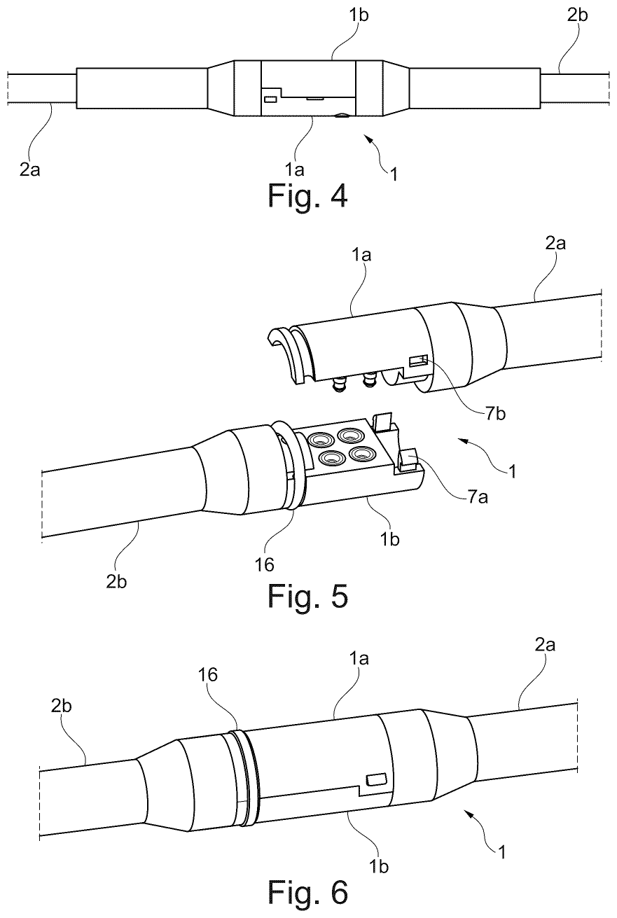

FIG. 4 shows a plug connector with two plug connector parts 1a, 1b in the connected state. In this embodiment, the longitudinal axis of the plug connector extends parallel to the longitudinal axes of the lines 2a, 2b that are connected to it. However, it is also possible that one or both longitudinal axes of the lines connected to the plug connector extend at an acute angle to the longitudinal axis of the plug connector. In this case, the longitudinal axes of the contact pole parts can be aligned for example perpendicularly or at an angle of between 30° and 90° to a longitudinal axis of at least one of the connected lines or to the longitudinal axis of the plug connector.

FIG. 5 shows a perspective view of a plug connector 1a, 1a, wherein the plug connector parts in this embodiment are held together on the one hand by a latching connection 7a, 7b and on the other hand by an elastic ring 16 which surrounds both plug connector parts.

FIG. 6 shows the plug connector parts 1a, 1b of FIG. 5 in the connected state.

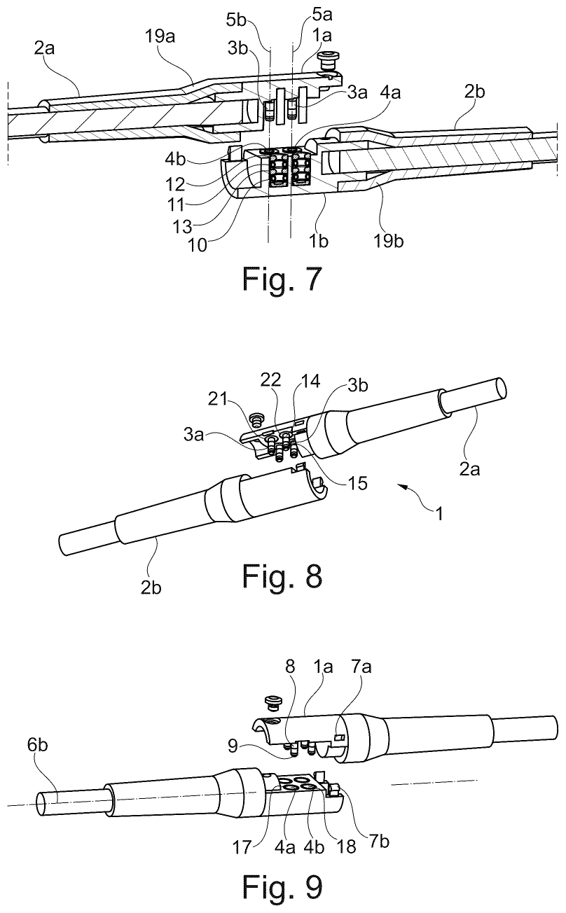

FIG. 7 shows a longitudinal cross-section of two plug connector parts showing an internal view of the contact sockets 4a, 4b. Inside the contact sockets 4a, 4b a plurality of annular spring contacts 10, 11 are shown in succession in the direction of the longitudinal axes 5a, 5b, each of which cooperates with contact surfaces 8, 9 of the contact pins 3a, 3b, which are shown in more detail in FIG. 9. Sealing rings 12, 13, which can be in the form of elastomer rings, are located between the annular spring contacts 10, 11. The elastomer rings 12, 13 each seal at sealing surfaces on the contact pins 3a, 3b which are arranged there as insulating sleeves between the contacts 8, 9 and are denoted in FIG. 8 as 14, 15.

FIG. 7 shows the lines 2a, 2b on both sides of the plug connector parts 1a, 1b, which also include the respective bend protection 19a, 19b. The bend protection 19a, 19b has a funnel-like extension which in each case comprises a cylindrical socket of the associated plug connector part.

In FIG. 8, annular grooves 21, 22 are provided at the points at which the contact pins 3a, 3b emerge from the plug connector part 1a, each of which concentrically surround a contact pin. Contact pole sealing rings 17, 18 engage in these grooves 21, 22 when the connector is plugged together and thus seal the cylindrical space around the contact pole parts in a fluid-tight manner. In the exemplary embodiment shown in FIG. 9, the contact pole sealing rings 17, 18 are integrated into the housing of the plug connector part 1b and protrude slightly over the edges of the contact sockets in the direction of the longitudinal axes of the contact pole parts. This ensures a reliable contact of the contact pole sealing rings and both plug connector parts.

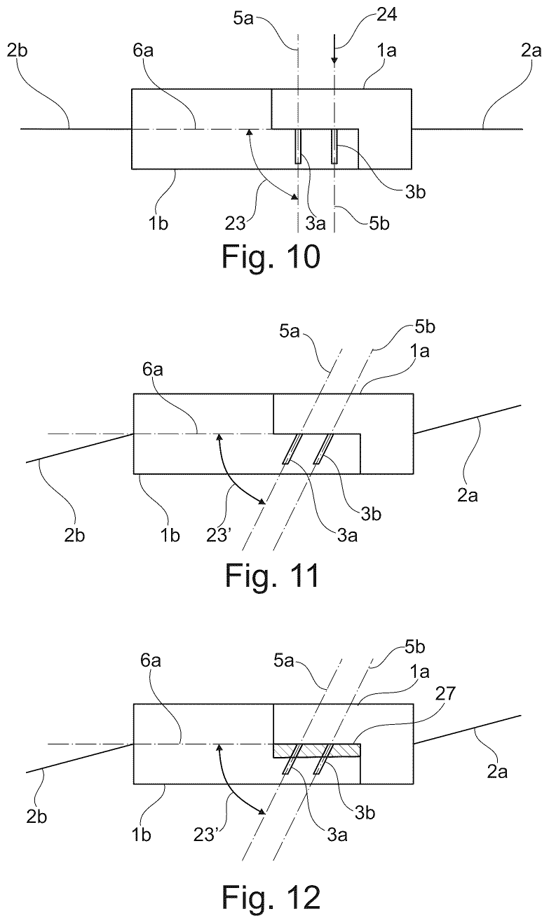

FIG. 10 shows schematically a plug connector in the connected state with a longitudinal axis 6a. The connected lines 2a, 2b also extend in the direction of the longitudinal axis 6a. The longitudinal axes 5a, 5b of the contact pins 3a, 3b extend perpendicular to the longitudinal axis 6a, i.e. the two longitudinal axes 6a and 5a, 5b form an angle 23 of 90 degrees. The plug-in direction for connecting the plug connector extends in the direction of the longitudinal axes of the contact pins or the contact poles and is denoted in FIG. 10 by 24.

In another possible embodiment according to FIG. 11, the mutually parallel longitudinal axes 5a, 5b of the contact pins 3a, 3b are not perpendicular to the longitudinal axis of a plug connector part, but form an angle 23′ with the longitudinal axis 6a, which can be from 30 degrees to 90 degrees, in particular also from 45 degrees to 90 degrees. This angle indication refers in each case to the smaller of the two angles, which is between a longitudinal axis of the contact poles and a longitudinal axis of a plug connector part or between a longitudinal axis of the contact poles and a longitudinal direction of one of the two lines connected to the plug connector, such that an insertion of the contact pins into the contact sockets in cases where the angle 23 is less than 90 degrees, also means that the two plug connector parts move closer to one another in their axial direction.

FIG. 12 shows a plug connector as shown in FIG. 11, wherein however the plug connector part 1b bears a sealing sleeve 27. When the plug connector parts 1a, 1b are plugged together, this is compressed between them in the plug-in direction, i.e. in the direction of the longitudinal axes 5a, 5b of the contact pole parts, and has openings and channels for receiving the contact pins 3a, 3b.

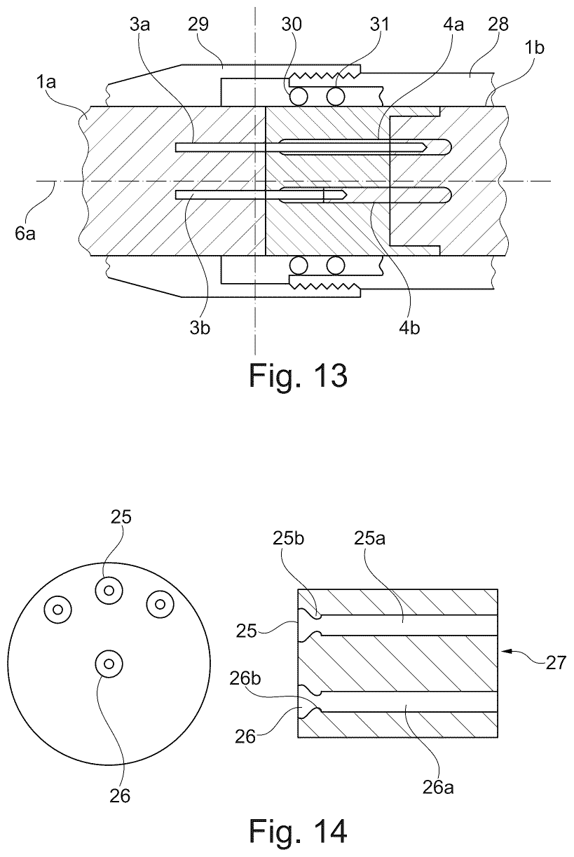

FIG. 13 shows a part of a plug connector in longitudinal cross-section, the plug connector parts 1a, 1b of which are plugged together in the direction of their common longitudinal axis 6a. In this case the plug connector parts can be cylindrically symmetrical in terms of their outer contour for example. The plug connector part 1a has two contact pins 3a, 3b, the contact pin 3a of which is depicted to be so long that it extends into a contact socket 4a of the plug connector part 1b. The contact socket 4a does not extend into the sealing sleeve 27, but ends axially in front of it.

The second contact pin 3b extends axially less far over the plug connector part 1a than the contact pin 3a and extends into the contact socket 4b, which extends axially from the plug connector part 1b into the sealing sleeve 27. These two examples are intended to represent various possible embodiments, in which all contact pins on the one hand and all contact sockets on the other hand can have the same form.

A sleeve 28 with an external thread is connected to the plug connector part 1b. A union nut 29 can be screwed onto this, whereby the two plug connector parts 1a, 1b can be pressed together axially.

In the course of such axial compression, the sealing sleeve 27 is also compressed axially, so that it expands in radial direction.

This can create or support fluid-tight bearing on the sealing rings 30, 31. In addition, the openings and channels inside the sealing sleeve can also be radially compressed which is explained further with reference to FIG. 14.

FIG. 14 shows on the right side a longitudinal cross-section of a sealing sleeve and on the left side a front view of a sealing sleeve.

The channels 32, 33 are shown in longitudinal cross section and are narrowed in the region of the openings 34, 35 by radially inwardly pointing sealing lips 32a, 33a. The narrowed points are dimensioned such that contact pins are in sliding contact with sealing lips on insertion and removal. In this way, any liquids adhering to the contact pins are wiped off as far as possible, so that the channels can be kept free of liquids.

1. An implantable electrical plug connector with two plug connector parts which can be joined together to make electrical contact, one of which is connected to a first line and the other of which is connected to a second line, wherein each plug connector part has at least one or at least two contact pole parts each in the form of a contact pin or a contact socket, which can be plugged together respectively with contact pole parts of the other plug connector part for making contact,
characterized in that the longitudinal axes of the contact pole parts extend at an angle of at least 30 degrees to a longitudinal axis of the first and/or second plug connector part.

2. An implantable electrical plug connector with two plug connector parts which can be joined together to make electrical contact, one of which is connected to a first line and the other of which is connected to a second line, wherein each plug connector part has at least one or at least two contact pole parts each in the form of a contact pin or a contact socket, which can be plugged together respectively with contact pole parts of the other plug connector part for making contact,
characterized in that the longitudinal axes of the contact pole parts extend at an angle of at least 30 degrees to a longitudinal extension direction of the first and/or second line.

3. The implantable electrical plug connector according to claim 1, characterized in that at least two contact pole parts are provided next to one another on each of the plug connector parts, which have longitudinal axes parallel to one another.

4. The implantable electrical plug connector according to claim 1, characterized in that at least one of the contact pole parts has a plurality of concentric contacts which are electrically insulated from one another.

5. The implantable electrical plug connector according to claim 4, characterized in that at least two of the concentric contacts respectively are offset relative to one another in a longitudinal direction of the respective contact pole part.

6. The implantable electrical plug connector according to claim 1, characterized in that the contact pins or the contact sockets each have one or more annular spring contacts.

7. The implantable electrical plug connector according to claim 5, characterized in that a sealing ring is arranged between two contacts in the direction of the longitudinal axes of the contact pole parts respectively.

8. The implantable electrical plug connector according to claim 1, characterized in that contact pole parts which engage with one another in the plugged-in state are arranged such that one or more scaling rings, which are assigned to one of the contact pole parts, bear in a sealing manner on sealing surfaces of the respective other contact pole part, wherein the sealing surfaces are formed in particular by cylindrical surfaces of electrically insulating sleeves.

9. The implantable electrical plug connector according to claim 1, characterized in that the two plug connector parts are held together by at least one elastic ring embracing both plug connector parts and/or a latching connection engaging in the direction of the longitudinal axes of the contact pole parts.

10. The implantable electrical plug connector according to claim 1, characterized in that each pair of contact pole parts is surrounded in the plugged-in state by an elastic contact pole scaling ring, which is arranged between the first plug connector part and the second plug connector part, and bears in a sealing manner on each of the plug connector parts, wherein in particular it is provided that the contact pole sealing rings are integrated respectively into one of the plug connector parts.

11. The implantable plug connector according to claim 1, wherein the longitudinal axes of the contact pole parts extend at an angle of at least 30 degrees, in particular at an angle of 90 degrees, to a longitudinal axis of the first and/or the second plug connector part or parallel to the longitudinal axis of the first and/or the second plug connector part, characterized in that one of the plug connector parts, which has at least one contact socket, has or supports on its side facing the other plug connector part when the plug connector is in a plugged-in state, a sealing sleeve, which is elastically deformable at least in some sections, in particular with at least one opening for receiving a contact pin.

12. The implantable plug connector according to claim 11, characterized in that the sealing sleeve consists of an elastomer.

13. The implantable plug connector according to claim 11, characterized in that one or more openings of the sealing sleeve open respectively into a channel for receiving a contact pin, wherein the channels extend respectively coaxially to contact sockets of the plug connector part which comprises the sealing sleeve and wherein at least one of the channels has a narrow point in the region of its opening.

14. The implantable plug connector according to claim 11, characterized in that the narrow points are formed respectively by inwardly pointing scaling lips in the channels.

15. The implantable plug connector according to claim 11, characterized in that the sealing sleeve in the plug connector can be compressed in axial direction or plug-in direction of the plug connector such that it expands in the directions perpendicular to the axial direction or plug-in direction of the plug connector.

16. The implantable plug connector according to claim 2, characterized in that at least two contact pole parts are provided next to one another on each of the plug connector parts, which have longitudinal axes parallel to one another.

17. The implantable plug connector according to claim 2, characterized in that at least one of the contact pole parts has a plurality of concentric contacts which are electrically insulated from one another.

18. The implantable plug connector according to claim 17, characterized in that at least two of the concentric contacts respectively are offset relative to one another in a longitudinal direction of the respective contact pole part.

19. The implantable plug connector according to claim 2, characterized in that the contact pins or the contact sockets each have one or more annular spring contacts.

20. The implantable electrical plug connector according to claim 2, characterized in that the two plug connector parts are held together by at least one elastic ring embracing both plug connector parts and/or a latching connection engaging in the direction of the longitudinal axes of the contact pole parts.
